(19) United States
(12) Patent Application Publication
(10) Pub. No.: US 2023/0105599 A1
(43) Pub. Date: Apr. 6, 2023

(54) Aqueous Formulations Containing Povidone Iodine for Effective Treatment and Prevention of Virus Infections

(71) Applicants: IVIEW Therapeutics, Inc., Cranbury, NJ (US); IVEW THERAPEUTICS (ZHUHAI) CO., LTD., Hengqin New Destrict, Zuha, Guangdong (CN)

(72) Inventor: Bo Liang, Doylestown, PA (US)

(73) Assignees: IVIEW Therapeutics, Inc., Cranbury, NJ (US); IVEW THERAPEUTICS (ZHUHAI) CO., LTD., Hengqin New Destrict, Zuha, Guangdong (CN)

(21) Appl. No.: 17/281,553

(22) PCT Filed: Mar. 29, 2021
(86) PCT No.: PCT/US2021/024577

§ 371 Date: Mar. 30, 2021

Related U.S. Application Data
(60) Provisional application No. 63/001,311, filed on Mar. 28, 2020.

(51) Int. Cl. A61K 33/18; A61K 31/4706; A61K 31/675; A61K 31/58; A61K 31/56; A61K 31/573; A61K 9/00; A61P 31/14; A61K 47/36; A61P 31/16

(52) U.S. Cl. CPC A61K 33/18; A61K 31/4706; A61K 31/675; A61K 31/58; A61K 31/56; A61K 31/573; A61K 9/0043; A61P 31/14; A61K 47/36; A61P 31/16

(57) ABSTRACT

The present invention provides aqueous formulations containing povidone iodine to be topically applied into a body cavity such as nose of a subject. The formulations of this invention are useful for treating a disease or symptom related to or caused by infection of COVID-19 virus or influenza virus H1N1.

### CROSS-REFERENCE TO RELATED APPLICATION

This application claims priority to U.S. Application No. 63/001,311, filed on Mar. 28, 2020, the contents of which are incorporated herein by reference in their entirety.

### BACKGROUND OF INVENTION

The 2019-20 coronavirus pandemic is an ongoing pandemic of coronavirus disease 2019 (COVID-19), caused by severe acute respiratory syndrome coronavirus 2 (SARS-CoV-2). As of Mar. 9, 2021, more than 118 million cases of COVID-19 infection had been reported in more than 190 countries and territories, resulting in more than 2.6 million deaths.

The virus is typically spread by close contact and via respiratory droplets produced when people cough or sneeze. People may also catch COVID-19 by touching a contaminated surface and then their eyes, nose, or mouth. It is most contagious when people are symptomatic, although spread may be possible before symptoms appear. The time between exposure and symptom onset is typically around five days, but may range from two to fourteen days. Common symptoms include fever, cough, and short of breath. Complications may include pneumonia and acute respiratory distress syndrome.

SARS-CoV which causes SARS, has a unique pathogenesis because it causes both upper and lower respiratory tract infections. It has been reported that SARS-CoV-2 uses the same cell entry receptor, ACE2, to infect humans, as SARS-CoV, so clinical similarity between the two viruses is expected, particularly in severe cases.

There is no known specific antiviral treatment for COVID-19, but development efforts are underway, including testing of existing medications. Primary treatment is symptomatic and supportive therapy. See, e.g., “Coronavirus Disease 2019 (COVID-19)”. Centers for Disease Control and Prevention. 11 Feb. 2020. Retrieved 23 Mar. 2020. Recommended preventive measures include hand washing, covering the mouth when coughing, maintaining distance from other people, and monitoring and self-isolation for people who suspect they are infected. Recently some vaccines have been approved against COVID-19 virus. However, it is still unknown how long vaccines can protect people. People who have been fully vaccinated should still take preventive measures. Additional, how effective the vaccines are against new variants of the virus is still unknown. Early data suggest the vaccines could be less effective against new strains of the virus. Though it is unclear that if the COVID-19 infection is airborne or through contact, it's urgent to develop effective infection protection and treatment strategies to prevent this pandemic spread. Therefore, developing safe and broad-spectrum povidone iodine-based products to protect people from COVID-19 infection and block the transmission through eye, nose and mouth and treat the infection in the early stage thus avoid getting into severe infection stage is in significant urgent need.

Povidone iodine (PVP-I) is a complex of polyvinylpyrrolidone and iodine. It is also called iodophor and contains 9-12% effective iodine. It is a powerful disinfectant with a broad spectrum of applications and is strongly effective against viruses, bacteria, fungi, and mold spores. It causes little irritation on skin and has low toxicity and lasting effect, and can be used safely and easily. PVP-I products have been used for the disinfection of various bacteria and viruses for years because of their strong bactericidal and antiviral activities, including both enveloped and non-enveloped viruses. See, e.g., Wood A, Payne D. J., Hosp Infect. 1998 April; 38(4):283-95; Kanagalingam J, Feliciano R, Hah J H, Labib H, Le T A, Lin J C., Int J Clin Pract. 2015 November; 69(11):1247-56; Sauerbrei A, Wutzler P., Lett Appl Microbiol. 2010 August; 51(2):158-63.

The virucidal activity is mainly due to the free iodine released from povidone-iodine. See, e.g., H. Wada et al., Biocontrol Sci. 2016; 21(1):21-7. Although PVP-I has shown a high virucidal efficacy against enveloped viruses, as well as against some nonenveloped human viruses (e.g., adenovirus and polyomavirus) (see, e.g., H. Kariwa et. al., Dermatology. 2006; 212 Suppl 1:119-23), it is unknown whether it would be effective in preventing or treating COVID-19 infection as many existing antiviral drugs have failed in treating COVID Given the differences between like many other drugs that have been proven effective in treating the same virus but did not work for COVID, there is no evidence that povidone iodine products can be effective to treat COVID and no PVP-I products have been studies as potential protection and prevent the spread of this new deliberating virus.

### BRIEF DESCRIPTION OF THE INVENTION

In one aspect, this invention is to develop safe and non-irritating povidone iodine compositions for the prevention and treatment of COVID-19 infection in the eye, nose and mouth. The povidone iodine compositions can be eye drops, nasal irrigation solution or nasal spray, or mouth drop, spray or wash. The concentrations of povidone iodine can range from 0.1% to 5%.

This invention is aimed to produce an in-situ gel formulation where the effective concentration of PVP-I is maintained by the equilibrium between solution PVP-I and the gel bound components resulting in a long lasting, less toxic pharmacological effect in the mucosa area in the eye, nose and mouth. This invention further describes the development of novel in-situ gel forming compositions.

Conventional liquid ocular formulations are rapidly eliminated from the precorneal area through lachrymation and nasolacrimal drainage, while a significant challenge of nasal drug development is to overcome the protective barriers of the nasal cavity without causing permanent tissue damage. The major problems that persist with nasal solutions are cleared off rapidly from nasal cavity. Such a problem can be overcome by using in-situ gel-forming drug delivery systems that exhibit sol-to-gel phase transitions due to a change in a specific physicochemical parameter in the cul-de-sac. The in-situ gel technology will greatly increase PVP-I's tissue retention time, thus greatly increase PVP-I's bioavailability and rendering long-acting efficacy against infective pathogens.

In one aspect, the present invention provides aqueous formulations for preventing or treating a disease or physical symptom related to or caused by infection of COVID-19 or influenza virus H1N1 in a subject in need thereof. Such a formulation includes water as solvent, a biocompatible polysaccharide dissolved in the aqueous solvent, and povidone iodine as a therapeutic agent, and the formation forms a gel upon topical application into an eye, nose or mouth of the subject.

In some embodiments, the concentration of povidone iodine in the formulations range from 0.1% to 5% [weight by weight (w/w) or weight by volume (w/v)], from 0.3% to 1% (w/w or w/v), or from 0.3% to 0.8% (w/w or w/v).

In some embodiments, the biocompatible polysaccharide includes deacetylated gellan gum, xanthan, sodium alginate, carrageenan, or any mixture thereof.

In some embodiments, the formulation further includes an anti-inflammatory agent, a steroid, an NSAID, or an antiviral or antimicrobial compound as a second therapeutic agent. Examples of a suitable antiviral or antimicrobial compound include hydroxychloroquine, chloroquine, and remdesivir. Examples of a suitable steroid include budesonide, mometasone, fluticasone, dexamethasone, and a salt, an ester, and/or any combination thereof. Particular examples of suitable steroid include budesonide, fluticasone, dexamethasone, a salt, an ester, and/or any combination thereof.

In some embodiments, the steroid is contained in the formulation at the concentration in the range of 0.05%-0.1%, 0.06%, 0.064%, or 0.08%.

In some embodiments, the aqueous formulation contains PVP-I at a concentration of 0.5%, 0.6%, 0.8%, or 1.0% (w/w or w/v).

The aqueous formulation of this invention can take the form of a solution, a suspension, an emulsion, an ointment, hydrogel, optionally with a drug delivery vehicle. On the other hand, the aqueous formulation can be used as eye drops, nasal irrigation solution, nasal spray, mouthwash or mouth spray.

Another aspect of the invention provides a method for preventing or treating a disease or physical symptom related to or caused by infection of COVID-19 or influenza virus H1N1 in a subject in need thereof. The method includes a step of administering a therapeutically effective amount of an aqueous formulation as described above upon topical application into an eye, nose or mouth of the subject. The aqueous formulation can be administered 1 to 24 times a day.

### DETAILED DESCRIPTION OF THE INVENTION

It's well known that major problems persist with nasal solutions as it is cleared off rapidly from the nasal cavity. The short contact time of water-soluble povidone iodine on nasal mucosa is undesirable and would necessitate frequent and multiple administration to maintain the virucidal efficacy, thereby limiting practicality and increasing medical burden for the patient. In addition, frequent dosing can lead to irritation and potential toxicity. Therefore, developing a safe, non-toxic and long-acting PVP-I nasal spray is an urgent medical need to protect people from SARS-CoV-2 infection, as well as to block the transmission through the nasal cavity.

The aqueous formulations of this invention contain water, a biocompatible polysaccharide dissolved in the aqueous solvent, and povidone iodine (PVP-I), and optionally other pharmaceutically acceptable carrier, wherein the formation forms a gel upon topical application into an eye, nose or mouth of a subject. These aqueous formulations are effective in preventing or treating a disorder or condition associated with or caused by COVID-19 infection. The formulations can be applied into a cavity of a subject (e.g., a mammal). The cavity can be eye, nose, or mouth.

The concentration of the PVP-I in the formulations of this invention may range from 0.1% to 5% [weight by weight (w/w) or weight by volume (w/v)], from 0.3% to 1% (w/w or w/v), or from 0.3% to 0.8% (w/w or w/v).

The compositions may further include (1) a topical anesthetic which relieves pain (2) a penetration enhancer which enhances the penetration of povidone-iodine into the tissues of the eye, nose or mouth, for example, Azone (laurocapram), a glucan sulfate such as dextran sulfate, cyclodextrin sulfate, and β-1,3-glucan sulfate (3) an antimicrobial preservative, which, for example, may be in a concentration of about 0.001% to 1.0% by weight; (4) a co-solvent or a nonionic surface agent-surfactant, which, for example, may be about 0.01% to 2% by weight; (5) viscosity increasing agent, which, for example, may be about 0.01% to 2% by weight; and (6) a cooling agent such as menthol, menthol derivatives including methone glycerin acetyl and methyl esters, carboxamides, methane glycerol ketals, alkylsubstituted ureas, sulfonamides, terpene analogs, furanones, and phosphine oxides; or camphor, and borneol, which can provide coolness sensation on the eye.

The compositions may further comprise other therapeutic agent such as anti-inflammatories, steroids, NSAIDs, and antiviral or antimicrobial compounds such as hydroxychloroquine, chloroquine, remdesivir (an antiviral medication containing nucleotide prodrug of an adenosine analog, sold under the brand name Veklury). The steroid maybe budesonide, mometasone, or fluticasone, or dexamethasone, or a salt, an ester, or any combination thereof.

The compositions are useful in the treatment of infections of the conjunctiva and cornea, nasal and sinus cavity, and mouth, particularly COVID-19 infection. In another embodiment, the invention is directed to a method for treating and/or prophylaxis of a disorder or a microorganism infection of at least one tissue of the eye, nose or mouth comprising the step of administering one of more doses of a composition, discussed above, to the eye, nose or mouth. In another embodiment, the invention is directed to a method for treating and/or prophylaxis of COVID-19 infection.

Suitable topical anesthetics for the compositions and methods of this invention include, at least, proparacaine, lidocaine, tetracaine or a derivative or combination thereof.

In any of the aqueous formulations of this invention that are suitable for topical administration, such as to the eye, nose, or mouth, the formulations are preferably prepared with 0.01-5.0% by weight of PVP-I at a pH of 3.5 to 6.0. This pH range may be achieved by the addition of acids/bases or buffers to the solution as needed.

While the precise regimen is left to the discretion of the clinician, it is recommended that the aqueous formulations of this invention be topically applied by placing one drop in each eye, or by irrigating or spray into nasal cavity, or by spray into mouth or mouth wash, 1 to 24 times daily.

### Antimicrobial Preservative

As an optional ingredient, suitable antimicrobial preservatives may be added to prevent multi-dose package contamination, though povidone-iodine will serve as self-preservative. Such agents may include benzalkonium chloride, thimerosal, chlorobutanol, methyl paraben, propyl paraben, phenylethyl alcohol, EDTA, sorbic acid, Onamer M, other agents known to those skilled in the art, or a combination thereof. Typically, such preservatives are employed at a level of from 0.001% to 1.0% by weight.

### Co-Solvents/Surfactants

The compositions of the invention may contain an optional co-solvent. The solubility of the components of the present aqueous formulations may be enhanced by a surfactant or other appropriate co-solvent in the formulations. Examples of such suitable co-solvents/surfactants include polysorbate 20, 60, and 80, polyoxyethylene/polyoxypropylene surfactants (e.g., Pluronic F-68, F-84 and P-103), cyclodextrin, tyloxapol, or a combination thereof. Typically, such co-solvents are employed at a level of from 0.01% to 2% by weight.

### Viscosity Agents

The compositions of the invention may contain an optional viscosity agent—that is, an agent that can increase viscosity. Viscosity increased above that of simple aqueous solutions may be desirable to increase absorption of the active compound in the ocular surface, in the nasal mucosa surface or sinus cavity, or in the mouth mucosa area, to decrease variability in dispensing the formulation, to decrease physical separation of components of a suspension or emulsion of the formulation and/or to otherwise improve the conformity of the formulation. Examples of such suitable viscosity builder agents include polyvinyl alcohol, polyvinyl pyrrolidone, methyl cellulose, hydroxy propyl methylcellulose, hydroxyethyl cellulose, carboxymethyl cellulose, hydroxy propyl cellulose, or a combination thereof. Such agents are typically employed at a level of from 0.01% to 2% by weight.

The invention is further elucidated with specific examples. It is understood that these examples are only used to describe the invention but not to intend to limit the scope of invention. The experimental methods with no specific conditions in the following examples, are usually prepared under conventional conditions, e.g., as described in the literature or according to the conditions suggested by the excipient manufacturer. Unless specifically stated, all percentages, ratios, proportions or fractions in this invention are calculated by weight by weight. Unless specifically defined in this invention, all professional and scientific terms used herein have the same meaning as well-trained personnel may be familiar with. In addition, any methods and materials similar or equivalent to those recorded in this invention can be applied to this invention. The preferred embodiments and materials described herein are used only for exemplary purposes.

### Example 1: Gel-Forming Aqueous Formulations Containing 1.0% PVP-I and Biocompatible Polysaccharide

Different polysaccharides such as deacetylated gellan gum (DGG), Xanthan gum, kappa-carrageenan, and alginate were screened to select the optimum ophthalmic gel forming matrix. The formulations with DGG in general exhibited in situ gelling ability under physiological conditions when DGG concentrations were optimized. DGG was chosen as the gel forming matrix in the formulation.

Sodium chloride, mannitol and tromethamine were mixed in water for injection. Weighted gellan gum was added and heated to 85-90° C. to dissolve the gellan gum. Sterile filter at temperature 80° C. into a compounding vessel, cool to room temperature to provide Solution 1. PVP-I was then dissolved in water for injection. Sterile filter into a sterile compounding vessel to provide Solution 2. The Sterilized Solution 2 was aseptically added to the Sterile filtered Solution 1 in a Class A area/room. The solutions were mixed and filled into bottles in a Class A area/room.

Three batches (180804, 180805, 180806) were filled into ambler glass screw cap vial, polyester plastic (PET) eye drop bottle and polypropylene (PP) plastic eye drop bottles to screen suitable container/closure for the drug product. The pH, available iodine content and viscosity of each formulation sample stored at 25° C./40% RH as accelerated storage condition for stability sampling at T=0, 15 days, 1 month (M), 2 M, 3 M and 6 M, and 2-8° C. as long term storage condition for stability sampling at T=0, 1, 3, 6, 9, 12, 18, 24 M are evaluated. The current stability study demonstrated the formulations were stable for at least 12 months with the study ongoing for 18 months and 24 months.

### Example 2: Gel-Forming Aqueous Formulations Containing 0.8% PVP-I and Polysaccharide

According to process provided in Example 1, gel-forming aqueous formulations containing 0.8% PVP-I were manufactured and put in stability chambers in which the formulations demonstrated stability at 2-8° C. for up to 12 M.

### Example 3: Aqueous Formulations Containing 0.6% PVP-I and Polysaccharide

According to process provided in Example 1, aqueous formulations containing 0.6% PVP-I were prepared and put in stability chambers in which the formulations demonstrated stability of up to 12 months at 2-8° C.

### Example 4: In Vitro Anti-Microbial Testing

Following the protocol described in the Journal of Clinical Microbiology, the eradication of Pseudomonas aeruginosa, MRSA, and Candida Parapsilosis at 15 seconds, 30 seconds, and 1 minutes was measured. The test article was made to use the formulations prepared in Examples 1-3 with tears (40:7) to mimic the condition in the eye for the in-situ gel formulation. Compounds demonstrated antimicrobial efficacy on the eradication of ocular isolates of Pseudomonas aeruginosa, MRSA, and Candida Parapsilosis at 15 s, 30 s, and 1 minute.

### Example 5: In-Vitro Anti-Viral Activity Assay

In a cytopathic effect study with human AE549 cells, an aqueous formulation containing 0.6% PVP-I demonstrated complete inactivation of Human Adenovirus Type 5 after 30±5 minutes liquid-liquid contact at 37±2° C. To determine whether contact with aqueous formulation of this invention containing 0.6% PVP-I inactivates (“kills”) virus, the aqueous formulation was mixed directly with virus for 30±5 minutes, then neutralized and the surviving virus was quantified. Neutralization controls showed that virus was effectively detected in the titer assay. Toxicity controls showed that titer plates were valid and no toxicity was observed in the 1/10 dilution of the test compound. The 70% ethanol was fully effective, and untreated virus controls were as expected. The undiluted aqueous formulation containing 0.6% PVP-I was an effective virucidal, and the 1/3.2 dilution (28% after virus added) had slight virucidal activity. The aqueous formulation containing 0.6% PVP-I exhibited complete inactivation of virus.

### Example 6: Irritation and Tolerability Study of Aqueous Formulations

Evaluation of the Tolerability of an aqueous formulation of this invention containing 0.6% PVP-I after Daily Topical Administration in NZW Rabbits has demonstrated that some signs of mild irritation initially associated with PVP-I, 0.6% or 0.3% (namely, discharge and congestion) resolved over time. Notably, at the later time points, animals treated with Aqueous formulation of this invention containing 0.6% PVP-I did not exhibit the mild congestion observed in the BSS control group at these time points, suggesting a possible protective effect. An aqueous formulation of this invention containing 0.6% PVP-I and 0.1% dexamethasone was used as a positive control. The congestion and swelling caused by the control did not appear to resolve over time. Moreover, the Positive control-treated animals developed mild conjunctival discharge at the later time points, a finding that was no seen in the BSS control group and may therefore reflect a true mildly irritating effect of the Positive Control.

### Example 7: Toxicology Studies of Aqueous Formulations

The purpose of this study was to determine the potential for ocular toxicity of an aqueous formulation of this invention containing PVP-I at two concentrations comparing each to the vehicle when administered by repeat topical ocular doses over a 7-day period to New Zealand white rabbits. No meaningful signs of ocular irritation or corneal staining were observed in the vehicle group (Group 1) during the study. Repeated topical dosing of each povidone iodine-containing formulations was associated brief behavioral signs of irritation after each drop (squinting and pawing of eyes), transient signs of mild ocular irritation, and persistent corneal staining. In Groups 2 (PVP-I 0.6%) and 3 (PVP-I 1.0%), mild to moderate ocular irritation was observed, characterized primarily by conjunctival involvement, and was more pronounced on Day 1 after the last dose, then lessened with time. A dose response with the irritation was observed whereby Group 3 (1.0% PVP-I) showed more signs of irritation, including transient corneal involvement, than Group 2 (0.6% PVP-I). In conclusion, the repeated topical administration of povidone iodine-containing gel formulations over 7-days was associated with mild and transient signs of ocular irritation. There were no associated histological findings. The results suggest that the aqueous formulations of this invention containing 0.6% PVP-I and 1.0% PVP-I (Group 2 and 3) were associated with transient irritation and mild toxicity limited to the superficial ocular tissues with a slight dose response suggested after Day 1. The lack of meaningful histological signs of corneal damage or inflammation support that the clinical observations were not associated with permanent changes after 7 days of dose administration.

### Example 8: In-Vivo Rabbit Anti-Viral Efficacy Study

The purpose of the study was to evaluate the efficacy of an aqueous formulation of this invention containing 0.6% PVP-I and an aqueous formulation of this invention containing 0.6% PVP-I and 0.1% dexamethasone suspension in a viral conjunctivitis model in New Zealand White rabbits. Twelve female New Zealand White rabbits were inoculated with Adenovirus Serotype 5 (Ad5) in both eyes via corneal scarification followed by topical viral administration. The animals were then treated with an aqueous formulation of this invention containing 0.6% PVP-I, a positive control article, 0.6% PVP-I and 0.1% dexamethasone suspension, or a negative control article, balanced salt solution (BSS), administered topically into both eyes once or twice daily for 10 days. Clinical ophthalmic examinations were performed on Days 1, 2, 3, 4, 7, and 10. Slit-lamp photographs were taken on Days 1, 3, 7, and 10. Clinical observations were performed daily. Both an aqueous formulation containing 0.6% PVP-I and 0.1% Dexamethasone (Suspension) and an aqueous formulation of this invention containing 0.6% PVP-I had ameliorating effects on the symptoms of Ad5-induced viral conjunctivitis, and that aqueous formulation of this invention containing 0.6% PVP-I produced more robust effects when administered twice daily rather than once daily. Statistical analysis further supported the conclusion that numerous ocular signs of inflammation were improved by treatment with the aqueous formulation of this invention containing 0.6% PVP-I and 0.1% dexamethasone suspension or containing only 0.6% PVP-I. Improvement over the vehicle group was found with both BID and QD treatment with the aqueous formulation of this invention containing 0.6% PVP-I for most findings.

It was surprisingly discovered that the aqueous formulations not only retained the drug on nasal mucosa longer and released the iodine in a sustained manner, thus enhancing the virucidal activity, it was helped deliver iodine into the sinus cavity, which is inside the nose and hard to reach by a conventional nasal spray solution that does not form a gel. With the sustained release of antiseptic on nasal mucosa and inside the sinus cavity, the nasal spray containing PVP-I will potentially kill the virus and block the virus transmission through the upper respiratory tract, thus avoiding getting the infection into the lower respiratory tract such as lungs. Further, the steroid contained in the aqueous formulations of this invention (e.g., budesonide) may have helped further improve the virucidal effect of PVP-I, and control the immune response especially for COVID infected patients which the inflammatory response may cause deadly effect.

### Example 9: Nasal Spray of Aqueous Formulations Containing 0.6% PVP-I

According to the process provided in Example 1, an aqueous formulation of this invention containing 0.6% PVP-I was prepared and dispersed into amber glass bottle and a nasal spray pump was installed.

### Example 10: Mouth Spray and Mouthwash of Aqueous Formulation

According to the process provided in Example 1, an aqueous formulation containing 0.6% PVP-I was prepared. It can be used as mouthwash directly or dispersed into polyester (PET) spray bottle as mouth spray.

### Example 11: Povidone Iodine Nasal Irrigation

An aqueous formulation of this invention containing 2.5% PVP-I was prepared and 5 mL of this 2.5% PVP-I formulation was diluted into 120 mL saline solution as 0.1% dilute povidone iodine solution to rinse the nasal cavity using NeilMed Sinus Rinse bottle.

### Example 12: Povidone Iodine/Budesonide Nasal Irrigation

An aqueous formulation containing 2.5% PVP-I and 0.005% Budesonide solution was prepared and 5 mL of such solution was diluted into 120 mL saline solution to produce an aqueous formulation containing 0.1% dilute povidone iodine and 0.25 mg budesonide solution to rinse the nasal cavity using NeilMed Sinus Rinse bottle.

### Example 13: Povidone Iodine/Budesonide Nasal Spray

Kelcogel gellan gum, sodium chloride, and tromethamine were weighed and added in water for injection in a compounding vessel. The mixture was vigorously stirred in an 85-90° C. water bath in Class D area; then the obtained solution was passed through 0.45 μm sterile filter and cooled to obtain the Solution 1. PVP-I was dissolved in water for injection in a compounding vessel at room temperature in Class D area. The obtained solution thus obtained was passed through 0.45 μm sterile filter to obtain Solution 2. Glycerin and budesonide were mixed together in appropriate amount of water for injection at the room temperature aseptically to obtain the Suspension 1 in Class C area. Solution 1 and Solution 2 and Suspension 1 were mixed together aseptically in a Class C area. The resultant obtained suspension was homogenized and mixed well. In process control, samples were taken and tested for appearance, pH, viscosity. The obtained suspension was then is transferred into sterilized glass bottles at about 10 g per 10 mL vial. The vials were capped with spray pump, then transferred to Class D area and installed with push button and cover. Then the bottles are transferred to common area for packaging. 0.8% and 0.6% PVP-I/budesonide 0.064% gel-forming suspension were made following the process.

### Example 14: In Vitro Antimicrobial Biofilm Testing

We have conducted in vitro microbiological biofilm study, which demonstrated the efficacy of 0.8% PVP-I/budesonide 0.064% gel-forming suspension against Pseudomonas aeruginosa with 8 Log reduction on average. The confirmatory in-vitro antimicrobial biofilm study has demonstrated anti-biofilm efficacy of 0.8% PVP-I/budesonide 0.064% gel-forming suspension against Pseudomonas aeruginosa, Staphylococcus aureus, and Candida albicans at 10 minutes and 1 hour.

### Example 15: Ex-Vivo Tissue Retention Study

The ex vivo nasal mucosa retention studies were conducted, comparing an aqueous formulation containing 0.8% PVP-I and budesonide 0.064% (Formulation C) with placebo control. The flow rate of artificial nasal fluid was set at 0.32 mL/min. The test articles were added onto porcine nasal mucosa using pipette and artificial nasal fluid continued for 1 minute. For aqueous formulation containing 0.8% PVP-I and 0.064% budesonide, the aqueous in-situ gel formulation was observed to stay on the mucosa surface. The amounts of PVP-I and budesonide in the eluents were measured. 13.91% of PVP-I was detected in the eluent of the placebo control, while PVP-I was not detected in the eluent of Formulation C. 33.29±10.75% of budesonide was detected in eluent of control, while only 3.14±0.58% of budesonide was found in eluent of formulation.

### Example 16: In Vivo Tissue Retention Study

The in vivo tissue retention studies in SD rats were conducted. An aqueous formulation in suspension containing 0.8% PVP-I and 0.064% budesonide and isotope labeled PVP-¹²⁵I and placebo control were compared to study the tissue retention. 10 μL of test articles were administered into right nostril. Rats were imaged under y radiation imaging at 0, 0.5, 1, 2, 3, 4, 5, 6, 8 and 24 hours after administration. Results showed that Formulation C (i.e., the aqueous formulation containing 0.8% PVP-I and 0.064% budesonide) showed higher tissue retention at 5 and 6 hours after administration (p<0.05).

Surprisingly, the gel-forming aqueous formulations of the present invention not only effectively killed virus by releasing iodine in a sustained manner, but also were able to reach and stay in sinuses where a typical nasal spray is not able to reach and exert the virus-killing effect of PVP-I.

### Example 17: Toxicology Studies

Buehler study: The Buehler study was conducted to determine potential allergic reactions caused by topical administration of 0.8% PVP-I/budesonide 0.064% gel-forming suspension by Buehler test (BT) in guinea pigs. Fifty Hartley guinea pigs (25 males and 25 females) were stratified by body weight and randomly assigned to the negative control group (sodium chloride solution) (10 animals), test article group Formulation C as a nasal spray (0.064% budesonide and 0.8% povidone iodine) (20 animals), excipient control group (Formulation C's excipients including gellan gum, sodium chloride, mannitol and tromethamine) (10 animals) and positive control group (1-chloro-2,4-dinitrobenzene, concentration of 1% at sensitization and 0.1% at challenge) (10 animals), with equal number of males and females in each group. Sensitization exposure phase: One day before administration, an area of about 2 cm×2 cm on the left side of the back of guinea pigs was depilated, smeared with 0.2 mL test article and covered with two layers of gauze and one layer of cellophane, which was sealed and fixed with non-irritating adhesive plaster for 6 hours. Then the dressing was removed and the skin was scrubbed with a cotton ball dipped in sodium chloride injection to remove the residual test article. The above procedure was repeated on Day 7 and Day 14. Challenge exposure phase: 14 days after the last induction (Day 28), a 2 cm×2 cm depilated area on the right side of the guinea pigs was applied with 0.2 mL test article using the same method as for the sensitization exposure (the animals was depilated 1 day before administration), and covered with two layers of gauze and one layer of cellophane for 6 hours, which was sealed and fixed with non-irritating adhesive plaster for 6 hours. Then the dressing was removed and the skin was scrubbed with a cotton ball dipped in sodium chloride injection to remove the residual test article. Clinical observations were performed daily during the study. Animals were weighed once on D-1, D14 and D28. Each animal was observed for skin erythema, edema and other abnormal reactions at 24 h and 48 h after removal of the covering during the challenge test. The erythema and edema were scored, and an animal would be judged as positive for skin allergic reaction based on a skin reaction score of 2. Incidence of allergic reaction was calculated.

Results: clinical observations during sensitization period: Negative control group, test article group and excipient control group: after sensitization, animals did not show abnormality in the general status, behavior, physical signs and skin of administration site. Post-challenge observations: at 24 h and 48 h after removing the covering in the challenge phase, no skin erythema and edema were observed in the guinea pigs of the negative control group, test article group and excipient control group, with a sensitization rate of 0%, indicating no skin allergic reaction. Body Weight: during the study, the body weights of animals in each group increased, without obvious abnormality. This shows that under the conditions of this study, Formulation C did not cause allergic skin reactions in guinea pigs.

### Example 18: Irritation/Tolerability Study

A 7-day nasal irritation study in rats was conducted using male Sprague-Dawley rats: Sprague Dawley CD® IGS rats to determine the potential of 0.8% PVP-I/budesonide 0.064% gel-forming suspension and 0.6% PVP-I/budesonide 0.064% gel-forming suspension (test substance) to produce nasal irritation. Eighteen healthy male rats (18) were selected for the test and equally distributed into six groups. Intranasal administration of aqueous formulation containing 0.8% PVP-I and 0.064% budesonide at dose levels of 25, 50 and 75 μL and 0.6% PVP-I and 0.064% budesonide gel-forming suspension at dose levels of 25 and 75 μL and saline control at dose levels of 75 μL were evaluated. The saline control or the test substance was administered into the right nostril via a 200 μL pipette twice daily (approximately 12 hours apart). The animals were observed at least once daily for viability, signs of gross toxicity, and behavioral changes, and weekly for a battery of detailed observations. Body weights were recorded two times during the acclimation period (including prior to study initiation on Day 1) and again prior to sacrifice. Individual food consumption was recorded to coincide with scheduled body weights. All animals were subjected to a gross necropsy of the upper respiratory tract and related sinuses at study termination (Day 7).

The results showed no signs of irritation and no abnormalities were detected following a gross necropsy of the upper respiratory tract and related sinuses. There were no mortalities during the course of the study and no test substance-related changes in body weight, body weight gain, and food consumption for the duration of the study. Under the conditions of this study and based on the endpoints evaluated, male Sprague Dawley rats are expected to tolerate dose levels of 75 μL of an aqueous formulation containing 0.8% PVP-I and 0.064% budesonide and an aqueous formulation containing 0.6% PVP-I and 0.064% budesonide.

### Example 19: 28-Day Repeat Dose Toxicity Study

The objective of this study was to evaluate the potential subchronic toxicity of an aqueous formulation containing 0.8% PVP-I and 0.064% budesonide in male and female rats likely to arise from repeated exposure to the test substance following intranasal administration over a 28-day test period. Potential reversibility of any possible test substance-related effects observed in the high dose group will also be evaluated after at least a 14-day recovery period. A no-observed-adverse-effect-level (NOAEL) is sought for each sex. A 28-day study was conducted in CRL: Sprague Dawley CD® IGS rats to determine the potential of 0.8% PVP-I/budesonide 0.064% gel-forming suspension (test substance) to produce subchronic toxicity. Sixty healthy rats (60) were selected for the test and equally distributed into four test groups and two recovery groups (control and high dose). Intranasal administration of the aqueous formulation containing 0.8% PVP-I and 0.064% budesonide at dose levels of 25, 50 and 75 μL and saline control at dose levels of 75 μL were evaluated. The saline control or the test substance was administered into the right nostril via a 200 μL pipette twice daily (approximately 12 hours apart). The animals were observed at least once daily for viability, signs of gross toxicity, and behavioral changes, and weekly for a battery of detailed observations. Body weights were recorded two times during the acclimation period (including prior to study initiation on Day 1) and weekly thereafter. Individual food consumption was recorded to coincide with scheduled body weights. All main study animals were subjected to a necropsy of the upper respiratory tract and related sinuses at study termination (Day 29). Thyroids and lungs were collected and weighed. There were no mortalities during the course of the study and no test substance-related changes in body weight, body weight gain, food consumption, thyroid weights and lung weights for the duration of the study. There were no macroscopic observations at terminal sacrifice considered attributable to the administration of 0.8% PVP-I/budesonide 0.064% gel-forming suspension. Notable necropsy findings were incidental and of no toxicological importance. A necropsy will be performed on recovery animals following a 14-Day recovery period.

Under the conditions of this study and based on the endpoints evaluated, Sprague Dawley rats are expected to tolerate dose levels of 75 μl of intranasal administration of 0.8% PVP-I/budesonide 0.064% gel-forming suspension.

### Example 20: In Vitro Virucidal Assay Against SARS-CoV-2

To determine whether contact with an aqueous formulation of this invention inactivates (“kills”) virus, the test formulation was mixed directly with virus for 30 seconds, 2 minute, and 10 minutes and the surviving virus was quantified.

Two of the aqueous formulations of this invention, Formulation A with 1.0% PVP-I and Formulation B with 0.6% PVP-I were prepared according the process described in Example 1 with the excipients including gellan gum, sodium chloride, mannitol and tromethamine. The two formulations were sent to Utah State University Antiviral Institute for virucidal assay against SARS-CoV-2. Test formulations were received in liquid form and held at 2-8° C. The formulations were diluted with artificial tears or artificial nasal fluid as follows: neat, 1/1.8, 1/3.2 and 1/10. Other controls were 45% ethanol and MEM solution. Virus solution was added to test dilution at 1/10 and incubated for 30 seconds, 2 minute, and 10 minutes at 37° C. As such, the final concentration of tested formulations were 90%, 50%, 28% and 9%. Following the incubation period, the samples were neutralized by a 1/10 dilution in test media containing 10% FBS. The surviving virus in each tube was determined using a standard endpoint dilution CCID₅₀ assay as follows. The samples were diluted 1/10 in series and each dilution added to 4 replicate columns of 96-well plates seeded with monolayers of Vero76 cells. Plates were incubated for 6 days at 37° C. with 5% CO₂ then scored for the presence or absence of cytopathic effect (CPE). Virus titer (CCID₅₀) was calculated using the Reed-Muench method (1948). The log reduction value (LRV) of the compound compared to the virus control was calculated. The entire procedure was repeated in triplicate. Toxicity controls were performed to show whether test compound toxicity would confound results by killing the cells in the absence of virus. Neutralization controls were performed to show that virus present could be detected in the presence of the test compound found on the titer plates.

Virus titers and LRV for Formulation A and Formulation B against SARS-CoV-2 are shown in Table 1. Compound toxicity was not observed at any concentration. In antiviral kinetics studies, a dose response was observed after treatment with both Formulation A and Formulation B. PVP-I formulations produced greater reduction in virus with increasing concentration and time of contact with the virus. Higher concentrations (0.9% PVP-I of Formulation A; 0.54% PVP-I of Formulation B) of the formulations completely inactivated SARS-CoV-2 virus, reducing titers below the level of detection. This was similar for the half concentration of both Formulation A (0.5%) and Formulation B (0.3%), which also reduced virus to near or below the level of detection. Lower concentrations (0.28% PVP-I of Formulation A; 0.17% PVP-I of Formulation B) also reduced the virus substantially. The lowest concentration (0.09% PVP-I of Formulation A; 0.05% PVP-I of Formulation B) of the formulations did not reduce virus significantly with increased contact time.

TABLE 1
Virucidal efficacy against Covid-19 virus after incubation with virus at 37° C..

| | Drug | PVP-I | | | |
|---|---|---|---|---|---|
| | Concen- tration | Concen- tration | Contact Time | Viral | |
| Sample | (%) | (%) | (min) | Titer^{a} | LRV^{b} |
| Formulation A | 90 | 0.9 | 0.5 | <0.67 ± 0.0 | 3.5 |
| Formulation A | 50 | 0.5 | 0.5 | 1.0 ± 0.3 | 3.2 |
| Formulation A | 28 | 0.28 | 0.5 | 2.0 ± 1.0 | 2.2 |
| Formulation A | 9 | 0.09 | 0.5 | 3.0 ± 0.4 | 1.2 |
| Formulation B | 90 | 0.54 | 0.5 | 1.1 ± 0.1 | 3.1 |
| Formulation B | 50 | 0.3 | 0.5 | 1.1 ± 0.3 | 3.1 |
| Formulation B | 28 | 0.17 | 0.5 | 1.2 ± 0,4 | 2.9 |
| Formulation B | 9 | 0.05 | 0.5 | 1.9 ± 1.0 | 2.3 |
| Ethanol | | | 0.5 | <1.7 ± 0.0 | 3.5 |
| Virus Control | | | 0.5 | 4.2 ± 0.4 | 0 |
| Formulation A | 90 | 0.9 | 2 | <0.67 ± 0.0 | 2.9 |
| Formulation A | 50 | 0.5 | 2 | <0.67 ± 0.0 | 2.9 |
| Formulation A | 28 | 0.28 | 2 | 1.0 ± 0.3 | 2.6 |
| Formulation A | 9 | 0.09 | 2 | 2.8 ± 0.4 | 0.8 |
| Formulation B | 90 | 0.54 | 2 | <0.67 ± 0.0 | 2.9 |
| Formulation B | 50 | 0.3 | 2 | <0.67 ± 0.0 | 2.9 |
| Formulation B | 28 | 0.17 | 2 | 0.8 ± 0.2 | 2.8 |
| Formulation B | 9 | 0.05 | 2 | 1.7 ± 0.6 | 1.9 |
| Ethanol | | | 2 | <1.7 ± 0.0 | 1.9 |
| Virus Control | | | 2 | 3.6 ± 0.1 | 0 |
| Formulation A | 90 | 0.9 | 10 | <0.67 ± 0.0 | 3.3 |
| Formulation A | 50 | 0.5 | 10 | <0.67 ± 0.0 | 3.3 |
| Formulation A | 28 | 0.28 | 10 | <0.67 ± 0.0 | 3.3 |
| Formulation A | 9 | 0.09 | 10 | 2.9 ± 0.4 | 1 |
| Formulation B | 90 | 0.54 | 10 | <0.67 ± 0.0 | 3.3 |
| Formulation B | 50 | 0.3 | 10 | <0.67 ± 0.0 | 3.3 |
| Formulation B | 28 | 0.17 | 10 | <0.67 ± 0.0 | 3.3 |
| Formulation B | 9 | 0.05 | 10 | 2.4 ± 0.1 | 1.6 |
| Ethanol | | | 10 | <0.67 ± 0.0 | 3.3 |
| Virus Control | | | 10 | 4.0 ± 0.3 | 0 |

^{a}Log10 CCID50 of virus per mL, mean of 3 replicates ± standard deviation.
^{b}LRV (log reduction value) is the reduction of virus compared to the virus control.

### Example 21: In Vitro Virucidal Assay Against Influenza Virus

Formulation B containing 0.6% PVP-I (for nasal spray) was tested for virucidal assay against influenza virus (Influenza Virus A H1N1, California July 2009). Formulation B was diluted with artificial nasal fluid as follows: neat, 1/1.8, 1/3.2 and 1/10. Other controls were 95% ethanol and MEM solution. Virus solution was added to test dilution at 1/10 and incubated for 30 seconds and 2 minutes at 37° C. And thus, the final concentration of solution tested were 90%, 50%, 28% and 9%. Following the incubation period, samples were neutralized by a 1/10 dilution in test media containing 10% FBS. The surviving virus in each tube was determined using a standard endpoint dilution CCID₅₀ assay as follows. Samples were diluted 1/10 in series and each dilution added to 4 replicate columns of 96-well plates seeded with 80-100% confluent MDCK cells. Plates were incubated for 6 days at 37° C. with 5% CO₂ then scored for the presence or absence of cytopathic effect (CPE). Virus titer (CCID₅₀) was calculated using the Reed-Muench method (1948). The log reduction value (LRV) of the compound compared to the virus control was calculated. The entire procedure was repeated in triplicate. Toxicity controls were performed to show whether test compound toxicity would confound results by killing the cells in the absence of virus. Neutralization controls were performed to show that virus present could be detected in the presence of the test compound found on the titer plates.

Virus titers and LRV for Formulation B against influenza are shown in Table 2. Compound toxicity was not observed at any concentration. In antiviral kinetics studies, a dose response was observed after treatment with Formulation B. PVP-I formulations produced greater reduction in virus with increasing concentration and time of contact with the virus. Higher concentrations (0.54% PVP-I of Formulation B) of the formulations completely inactivated SARS-CoV-2 virus, reducing titers below the level of detection. This was similar for the half concentration of Formulation B (0.3%), which also reduced virus to near or below the level of detection. Lower concentrations (0.17% PVP-I of Formulation B) also reduced the virus substantially. The lowest concentration (0.05% PVP-I of Formulation B) of the formulations did not reduce virus significantly with increased contact time.

TABLE 2
Virucidal efficacy against influenza virus after incubation with virus at 37° C..

| | Drug | PVP-I | | | |
|---|---|---|---|---|---|
| | Concen- tration | Concen- tration | Contact Time | Viral | |
| Sample | (%) | (%) | (min) | Titer^{a} | LRV^{b} |
| Formulation B | 90 | 0.54 | 0.5 | <0.67 | >5.3 |
| Formulation B | 50 | 0.3 | 0.5 | 2.5 | 3.5 |
| Formulation B | 28 | 0.17 | 0.5 | 3 | 3.0 |
| Formulation B | 9 | 0.05 | 0.5 | 5.67 | 0.33 |
| Ethanol | | | 0.5 | 4.5 | 1.5 |
| Virus Control | | | 0.5 | 6.0 | — |
| Formulation B | 90 | 0.54 | 2 | <0.67 | >6.0 |
| Formulation B | 50 | 0.3 | 2 | <0.67 | >6.0 |
| Formulation B | 28 | 0.17 | 2 | 2.7 | 4.0 |
| Formulation B | 9 | 0.05 | 2 | 5.5 | 1.2 |
| Ethanol | | | 2 | 2.3 | 4.4 |
| Virus Control | | | 2 | 6.7 | — |

^{a}Log10 CCID50 of virus per mL.
^{b}LRV (log reduction value) is the reduction of virus compared to the virus control.

### Example 22. Test of Formulations in Human with COVID-19 Virus Infection

The aqueous formulations of this invention containing DGG and polysaccharide (with 1.0% or 0.6% PVP-I, optionally with budesonide or fluticasone) are used in clinical trials to treat patients infected with COVID-19 in the early stage of the diseases or symptoms to lessen the infection thus preventing patient getting into severe stage. The vehicle-controlled trial is conducted in Canada with primary end-point on symptom improvement assessed by nasal SNOT-22 scores and secondary end-point as viral eradication of saliva samples of patients dosed with the drugs vs. placebo. These formulations unexpectedly undergo sol-gel transformation and form gel in situ upon application or instillation on the mucosal surface (likely due to the presence of sodium, potassium and calcium ions in the mucus) and provide sustained release of PVP-I from the gel, therefore imparting a long-acting virucidal effect on the COVID-19 virus and resulting in effective treatment and prevention of disease or symptoms related to or caused by COVID-19 infection. This is a significant and highly desirable improvement over application of a conventional aqueous solution for the same purpose.

The invention has been described herein by reference to certain preferred embodiments. However, as obvious variations thereof will become apparent to those skilled in the art, the invention is not to be considered as limited thereto. All patents, patent applications, and references cited anywhere is hereby incorporated by reference in their entirety.

1. An aqueous formulation for preventing or treating a disease or physical symptom related to or caused by infection of COVID-19 or influenza virus H1N1 in a subject in need thereof, comprising water as solvent, a biocompatible polysaccharide dissolved in the aqueous solvent, and povidone iodine as a therapeutic agent, wherein the formation forms a gel upon topical application into an eye, nose or mouth of the subject.

2. The aqueous formulation of claim 1, wherein the concentration of povidone iodine in the formulation range from 0.1% to 5% [weight by weight (w/w) or weight by volume (w/v)], from 0.3% to 1% (w/w or w/v), or from 0.3% to 0.8% (w/w or w/v).

3. The aqueous formulation of claim 1, wherein the biocompatible polysaccharide comprise deacetylated gellan gum, xanthan, sodium alginate, carrageenan, or any mixture thereof.

4. The aqueous formulation of claim 3, further comprising an anti-inflammatory agent, a steroid, an NSAID, or an antiviral or antimicrobial compound as a second therapeutic agent.

5. The aqueous formulation of claim 4, wherein the antiviral or antimicrobial compound is hydroxychloroquine, chloroquine, or remdesivir; and the steroid is budesonide, mometasone, fluticasone, or dexamethasone, or a salt, an ester, or any combination thereof.

6. The aqueous formulation of claim 5, wherein the steroid is budesonide, fluticasone, or dexamethasone, or a salt, an ester, or any combination thereof.

7. The aqueous formulation of claim 6, wherein the steroid is contained in the formulation at the concentration in the range of 0.05%-0.1%.

8. The aqueous formulation of claim 6, wherein the steroid is contained in the formulation at the concentration of 0.060%, 0.064%, or 0.080%.

9. The aqueous formulation of claim 8, wherein the formulation contains PVP-1 at a concentration of 0.5%, 0.6%, 0.8%, or 1.0% (w/w or w/v).

10. The aqueous formulation of claim 9, wherein the formulation takes the form of a solution, a suspension, an emulsion, an ointment, hydrogel, optionally with a drug delivery vehicle.

11. The aqueous formulation of claim 10, wherein the aqueous formulation is used as eye drops, nasal irrigation solution, nasal spray, mouthwash or mouth spray.

12. A method for preventing or treating a disease or physical symptom related to or caused by infection of COVID-19 or influenza virus H1N1 in a subject in need thereof, comprising a step of administering a therapeutically effective amount of an aqueous formulation of claim 1 upon topical application into an eye, nose or mouth of the subject.

13. The method of claim 12, wherein the aqueous formulation is administered 1 to 24 times a day.

14. The method of claim 12, wherein the concentration of povidone iodine in the formulation range from 0.1% to 5% [weight by weight (w/w) or weight by volume (w/v)], from 0.3% to 1% (w/w or w/v), or from 0.3% to 0.8% (w/w or w/v).

15. The method of claim 14, wherein the biocompatible polysaccharide comprise deacetylated gellan gum, xanthan, sodium alginate, carrageenan, or any mixture thereof.

16. The method of claim 15, wherein the formulation further comprises an anti-inflammatory agent, a steroid, an NSAID, or an antiviral or antimicrobial compound as a second therapeutic agent.

17. The method of claim 16, wherein the antiviral or antimicrobial compound is hydroxychloroquine, chloroquine, or remdesivir; and the steroid is budesonide, mometasone, fluticasone, or dexamethasone, or a salt, an ester, or any combination thereof.

18. The method of claim 16, wherein the steroid is budesonide, fluticasone, or dexamethasone, or a salt, an ester, or any combination thereof.

19. The method of claim 16, wherein the steroid is contained in the formulation at the concentration of 0.060%, 0.064%, or 0.080%.

20. The method of claim 19, wherein the formulation contains PVP-1 at a concentration of 0.5%, 0.6%, 0.8%, or 1.0% (w/w or w/v).
